SEQUENCE LISTING (4 sequences)

SEQ ID NO: 1 - DNA, 1820 bp, Corynebacterium glutamicum
  CDS (250)..(1587)
tacgagcggagaaatttgggctgttttcttgccctgacatgcgaacacaaaaggtaatattaattttctgtgaggggagtcatgaaatctgctttttgcgtcacaggaaaaagtttttcataattttccgaatcgtccgcggatagcattttaagcttcgtcggcaacgatcacaccacgttaaatttcgaccatcaatgacggtggccgttttttggcgaatccccaaaaaggtcgaagaggaagttcatggattcaaacacagaatcttcaagtgttgaggtcaaaaacgaacacattaaagttcaaaagccgccgaagaaggaccgcactcactggctctacattgcggtcattatcgcattgattggcggtattaccctaggcctgatttcaccggagttgggcaaagaattcaagattttgggcaccatgtttgtgtccttgatcaagatgattatcgctccagttattttctgcaccatcgtcatcggaatcggttcagtcaaggcagcggcaacagtcggacgcgctggtggcatcgcccttgcgtacttcatcacgatgtccacattcgcactcgcagttggcctgctagtcggtaacttcatccagccaggtagcggactgaacatctcagttgatgaagaatcttcattcgcatccacagagagcagccctgaaggactcttgggattcatccactcgatcatccctgaaacgttcttctctgcatttactgatggttcggtgctgcaggtactgttcatcgccatcctcgtgggctttgcagctcagtcgatgggtgaaaagggacagcccatccttgatttcgtatcccatctgcagaagctcatcttcaagattttgaactggattctgtggctcgccccagtcggtgcattcggtgcaatggccggcgtcgttggcgaaacaggctttgatgccgttgttcagctcggtattttgatcctcgccttttacgtcacctgcgtgatcttcatctttggcgtgctgggcgccgtactgaaggtgttcaccggcgtgaatatcttcaagctggtcaagtaccttgccaaggaattcctgctgatctttgctacctcatcctctgaatctgccttgccaaacctcatgcgcaagatggaacacatcggtgtggctaaaccaaccgtcggaatcgtggtcccaaccggctattccttcaacttggacggcaccgcaatttacctcaccatggcatctatcttcattgccgacgcgatgaatatgccgatgagcctcggcgagcaggtcggtctgcttgtcttcatgatcatcgcatccaagggcgctgctggtgtctcgggtgccggtattgcaacgttggctgccggattgtcttcacaccgcccagaacttctgcacggcgttgacgtgattgtgggcatcgataaattcatgtctgaagcccgcgcactaaccaacttcgccggaaactccgtggcaacactgctggtcggcaagtggactggcaccgtggacatgaaccaagtccatgacgttttgaatggaaaatctccatttgtggagttagaagaagaccactagttttcaacaggacgacaacggccggacatgcgacaatacaatgcatgtccggccgtcttcttgtttcagtttctagtattttcgaccagacccgatcggcggctgacaggctcatttcagacctgcgagccgacggcatcgaggtctcattacttgtcgcaccccgcatcgatggggactggcgtctcgccaaagacaaagggaccctcgcgtggatggaacaacaacgc

SEQ ID NO: 2 - protein, 446 aa, Corynebacterium glutamicum
MDSNTESSSVEVKNEHIKVQKPPKKDRTHWLYIAVIIALIGGITLGLISPELGKEFKILGTMFVSLIKMIIAPVIFCTIVIGIGSVKAAATVGRAGGIALAYFITMSTFALAVGLLVGNFIQPGSGLNISVDEESSFASTESSPEGLLGFIHSIIPETFFSAFTDGSVLQVLFIAILVGFAAQSMGEKGQPILDFVSHLQKLIFKILNWILWLAPVGAFGAMAGVVGETGFDAVVQLGILILAFYVTCVIFIFGVLGAVLKVFTGVNIFKLVKYLAKEFLLIFATSSSESALPNLMRKMEHIGVAKPTVGIVVPTGYSFNLDGTAIYLTMASIFIADAMNMPMSLGEQVGLLVFMIIASKGAAGVSGAGIATLAAGLSSHRPELLHGVDVIVGIDKFMSEARALTNFAGNSVATLLVGKWTGTVDMNQVHDVLNGKSPFVELEEDH

SEQ ID NO: 3 - DNA, 28 bp, Corynebacterium glutamicum
caggtaccaaggtcgaagaggaagttca

SEQ ID NO: 4 - DNA, 28 bp, Corynebacterium glutamicum
gatctagacggccggacatgcattgtat

(19) United States
(12) Patent Application Publication
(10) Pub. No.: US 2002/0106759 A1
(43) Pub. Date: Aug. 8, 2002

(54) Nucleotide sequences coding for the dctA gene

(76) Inventors: Mike Farwick, Bielefeld (DE); Klaus Huthmacher, Gelnhausen (DE); Brigitte Bathe, Salzkotten (DE); Thomas Hermann, Bielefeld (DE); Walter Pfefferle, Halle (DE)

Correspondence Address: SMITH, GAMBRELL & RUSSELL, LLP, 1850 M STREET, N.W., SUITE 800, WASHINGTON, DC, 20036, US

(21) Appl. No.: 09/951,780

(22) Filed: Sep. 14, 2001

(30) Foreign Application Priority Data
Sep. 19, 2000 (DE) 100 46 230.8
Jul. 5, 2001 (DE) 101 32 724.2

(51) Int. Cl. C12P 13/08; C07H 21/04; C12N 9/10; C12N 1/21; C12P 21/02; C12N 15/74

(52) U.S. Cl. 435/115; 435/69.1; 435/252.3; 435/193; 435/320.1; 536/23.2

(57) ABSTRACT

The invention relates to an isolated polynucleotide having a polynucleotide sequence which codes for the dctA gene, and a host-vector system having a coryneform host bacterium in which the dctA gene is present in attenuated form and a vector which carries at least the dctA gene according to SEQ ID No 1, and the use of polynucleotides which comprise the sequences according to the invention as hybridization probes.

### BACKGROUND OF THE INVENTION

The invention provides nucleotide sequences from coryneform bacteria coding for the dctA gene and a process for the fermentative preparation of amino acids using bacteria in which the dctA gene is enhanced. All references cited herein are expressly incorporated by reference. Incorporation by reference is also designated by the term “I.B.R.” following any citation.

L-amino acids, in particular lysine, are used in human medicine and in the pharmaceutical industry, in the foodstuffs industry and very particularly in animal nutrition.

It is known that amino acids can be prepared by the fermentation of strains of coryneform bacteria, in particular Corynebacterium glutamicum. Due to the importance of this area, constant efforts are made to improve the method of preparation. Process improvements may relate to fermentation technology such as, for example, stirring and supplying with oxygen, or the composition of the nutrient media such as, for example, the sugar concentration during fermentation, or working up to the product form by, for example, ion exchange chromatography, or the intrinsic performance properties of the microorganism itself.

To improve the performance properties of these microorganisms, the methods of mutagenesis, selection and mutant choice are applied. In this way, strains are obtained which are resistant to antimetabolites or are auxotrophic for regulatorily significant metabolites and which produce amino acids.

For some time now, the methods of recombinant DNA engineering have also been used for the strain improvement of L-amino acid producing strains of Corynebacterium, by amplifying the individual amino acid biosynthesis genes and examining the effects on amino acid production.

The invention provides new measures for the improved fermentative preparation of amino acids.

### BRIEF SUMMARY OF THE INVENTION

Wherever L-amino acids or amino acids are mentioned in the following, this is intended to mean one or more amino acids, including their salts, chosen from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophane and L-arginine. L-lysine is particularly preferred.

When L-lysine or lysine is mentioned in the following, this is intended to mean not only the bases but also salts such as e.g. lysine monohydrochloride or lysine sulfate.

The invention provides a polynucleotide isolated from coryneform bacteria and containing a polynucleotide sequence coding for the dctA gene, chosen from the group

a) a polynucleotide which is at least 70% identical to a polynucleotide which codes for a polypeptide which contains the amino acid sequence in SEQ ID No. 2,

b) a polynucleotide which codes for a polypeptide which contains an amino acid sequence which is at least 70% identical to the amino acid sequence in SEQ ID No. 2,

c) a polynucleotide which is complementary to the polynucleotides in a) or b), and

d) a polynucleotide containing a sequence of at least 15 consecutive nucleotides from the polynucleotide sequence in a), b) or c),

wherein the polypeptide preferably has the activity of the C4 dicarboxylate transport protein.

The invention also provides the polynucleotide mentioned above, wherein it is preferably a replicable DNA containing:

| | |
|---|---|
| (i) | the nucleotide sequence given in SEQ ID No. 1, or |
| (ii) | at least one sequence which corresponds to |
| | sequence (i) within the range of degeneracy of |
| | the genetic code, or |
| (iii) | at least one sequence which hybridises with |
| | sequences which are complementary to sequences |
| | (i) or (ii), and optionally |
| (iv) | functionally neutral sense mutations in (i). |

The invention also provides:

a replicable polynucleotide, in particular DNA, containing the nucleotide sequence shown in SEQ ID No.1;

a polynucleotide which codes for a polypeptide which contains the amino acid sequence shown in SEQ ID No. 2;

a vector, containing the polynucleotide according to the invention, in particular a shuttle vector or plasmid vector, and

coryneform bacteria which contain the vector or in which the dctA gene is enhanced.

The invention also provides polynucleotides which consist substantially of a polynucleotide sequence which are obtainable by the screening, by means of hybridization, of a suitable gene library from a coryneform bacterium which contains the complete gene or a part thereof, with a probe which contains the sequence in the polynucleotide according to the invention in accordance with SEQ ID No.1 or a fragment thereof and isolating the polynucleotide sequence mentioned.

### BRIEF DESCRIPTION OF THE FIGURES

FIG. 1: Map of the plasmid pEC-XK99E

FIG. 2: Map of the plasmid pEC-XK99EdctAb1ex

The abbreviations and names used are defined as follows:

| | |
|---|---|
| Kan: | Kanamycin resistance gene aph(3′)-IIa from |
| | Escherichia coli |
| HindIII | Restriction site of restriction enzyme |
| | HindIII |
| XbaI | Restriction site of restriction enzyme XbaI |
| KpnI | Restriction site of restriction enzyme KpnI |
| Ptrc | trc promoter |
| T1 | Termination region T1 |
| T2 | Termination region T2 |
| Per | Replication effector per |
| Rep | Replication region rep of plasmid pGA1 |
| LacIq | laclq repressor of lac operon from |
| | Escherichia coli |
| DctA | cloned dctA gene |

### DETAILED DESCRIPTION OF THE INVENTION

Polynucleotides which contain sequences in accordance with the invention are suitable as hybridization probes for RNA, cDNA and DNA, in order to isolate nucleic acids or polynucleotides or genes of full length which code for the C4 dicarboxylate transport protein, or in order to isolate nucleic acids or polynucleotides or genes which exhibit a high similarity to the sequence in the dctA gene. They may also be used as probes for so-called arrays, micro-arrays or DNA chips in order to detect, to analyze and to determine the corresponding polynucleotides.

Furthermore, polynucleotides which contain the sequences in accordance with the invention are suitable as primers, with the aid of which, and using the polymerase chain reaction (PCR), the DNA of genes which code for the C4 dicarboxylate transport protein can be prepared.

Those oligonucleotides which are used as probes or primers contain at least 25, 26, 27, 28, 29 or 30, preferably at least 20, 21, 22, 23 or 24, very particularly preferably at least 15, 16, 17, 18 or 19 consecutive nucleotides. Oligonucleotides with a length of at least 31, 32, 33, 34, 35, 36, 37, 38, 39 or 40, or at least 41, 42, 43, 44, 45, 46, 47, 48, 49 or 50 nucleotides are also suitable. Optionally, oligonucleotides with a length of at least 100, 150, 200, 250 or 300 nucleotides are also suitable.

“Isolated” means separated from its natural surroundings.

A “polynucleotide” generally refers to polyribonucleotides and polydeoxyribonucleotides, wherein these may be non-modified RNA or DNA or modified RNA or DNA.

Polynucleotides according to the invention include a polynucleotide in accordance with SEQ ID No. 1 or a fragment prepared therefrom and also those which are at least 70% to 80%, preferably at least 81% to 85%, particularly preferably at least 86% to 90%, and very particularly preferably at least 91%, 93%, 95%, 97% or 99% identical to the polynucleotide in accordance with SEQ ID No. 1 or a fragment prepared therefrom.

“Polypeptides” are understood to be peptides or proteins which contain two or more amino acids linked via peptide bonds.

Polypeptides according to the invention include a polypeptide in accordance with SEQ ID No. 2, in particular those with the biological activity of the C4 dicarboxylate transport protein and also those which are at least 70% to 80%, preferably at least 81% to 85%, particularly preferably at least 86% to 90%, and very particularly preferably at least 91%, 93%, 95%, 97% or 99% identical to the polypeptide in accordance with SEQ ID No. 2 and have the activity mentioned above.

Furthermore, the invention provides a process for the fermentative preparation of amino acids chosen from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophane and L-arginine, using coryneform bacteria, in particular those which already produce amino acids and in which the nucleotide sequences coding for the dctA gene are enhanced, in particular overexpressed.

In this context, the expression “enhancement” describes the increase in intracellular activity of one or more enzymes (proteins) in a microorganism which are coded by the corresponding DNA, for example by increasing the copy number for the gene or genes, by using a strong promoter or by using a gene or allele which codes for a corresponding enzyme (protein) with a high activity and optionally by combining these measures.

As a result of the enhancement measures, in particular overexpression, the activity or concentration of the corresponding protein is generally increased by at least 10%, 25%, 50%, 75%, 100%, 150%, 200%, 300%, 400% or 500%, with a maximum up to 1000% or 2000%, with respect to the initially used microorganism.

Microorganisms which are provided by the present invention can produce L-amino acids from glucose, saccharose, lactose, fructose, maltose, molasses, starch, cellulose or from glycerine and ethanol. They are representatives of coryneform bacteria, in particular of the genus Corynebacterium. From among the genus Corynebacterium, the species Corynebacterium glutamicum has to be mentioned in particular, this being recognized by a person skilled in the art for its ability to produce L-amino acids.

Suitable strains of the genus Corynebacterium, in particular of the species Corynebacterium glutamicum (C. glutamicum), are in particular the known wild type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

and L-amino acid-producing mutants or strains prepared therefrom.

The new dctA gene coding for the enzyme C4 dicarboxylate transport protein was isolated from C. glutamicum.

In order to isolate the dctA gene, or also other genes, from C. glutamicum, a gene library from this microorganism is first compiled in Escherichia coli (E. coli). The compilation of gene libraries is described in generally known textbooks and manuals. The text book by Winnacker: Gene und Klone, Eine Einführung in die Gentechnologie (Verlag Chemie, Weinheim, Germany, 1990) I.B.R., or the manual by Sambrook et al.: Molecular Cloning, A Laboratory Manual (Cold Spring Harbor Laboratory Press, 1989) I.B.R. may be mentioned as examples. A very well-known gene library is that of the E. coli K-12 strain W3110, which was compiled by Kohara et al. (Cell 50, 495-508 (1987)) I.B.R. in λ-vectors. Bathe et al. (Molecular and General Genetics, 252:255-265, 1996) I.B.R. describe a gene library from C. glutamicum ATCC13032, which was compiled with the aid of the cosmid vector SuperCos I (Wahl et al., 1987, Proceedings of the National Academy of Sciences USA, 84:2160-2164 I.B.R.) in E. coli K-12 strain NM554 (Raleigh et al., 1988, Nucleic Acids Research 16:1563-1575 I.B.R.).

Again, Börmann et al. (Molecular Microbiology 6(3), 317-326 (1992)) I.B.R. describe a gene library from C. glutamicum ATCC13032 obtained using the cosmid pHC79 (Hohn and Collins, Gene 11, 291-298 (1980) I.B.R.).

To prepare a gene library from C. glutamicum in E. coli, plasmids such as pBR322 (Bolivar, Life Sciences, 25, 807-818 (1979) I.B.R.) or pUC9 (Vieira et al., 1982, Gene, 19:259-268 I.B.R.) may also be used. Particularly suitable hosts are those E. coli strains which are restriction and recombination defective. An example of these is the strain DH5αmcr which was described by Grant et al. (Proceedings of the National Academy of Sciences USA, 87 (1990) 4645-4649) I.B.R. The long DNA fragments cloned with the aid of cosmids are then again subcloned in suitable vectors commonly used for sequencing and then sequenced, as is described e.g. in Sanger et al. (Proceedings of the National Academy of Sciences of the United States of America, 74:5463-5467, 1977) I.B.R.

The DNA sequences obtained may then be examined using known algorithms or sequence analysis programs such as e.g. the one from Staden (Nucleic Acids Research 14, 217-232(1986)) I.B.R., the one from Marck (Nucleic Acids Research 16, 1829-1836 (1988)) I.B.R. or the GCG program from Butler (Methods of Biochemical Analysis 39, 74-97 (1998)) I.B.R.

The new DNA sequence from C. glutamicum, coding for the dctA gene, was found in this way and, as SEQ ID No. 1, is a constituent of the present invention. Furthermore, the amino acid sequence for the corresponding protein was derived from the available DNA sequence using the methods described above. SEQ ID No. 2 gives the amino acid sequence in the dctA gene product which is obtained.

Coding DNA sequences which are produced from SEQ ID No. 1 by the degeneracy of the genetic code are also a constituent of the present invention. In the same way, DNA sequences which hybridize with SEQ ID No. 1 or parts of SEQ ID No. 1, are a constituent of the invention. Furthermore, in the specialist field, conservative amino acid replacements, such as e.g. replacing glycine by alanine or aspartic acid by glutamic acid, in proteins are known as sense mutations which do not lead to any fundamental change in the activity of the protein, i.e. they are functionally neutral. Furthermore, it is known that changes at the N-terminal and/or C-terminal of a protein do not substantially impair its function and may even stabilize it. A person skilled in the art may find information about this, inter alia, in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in well-known textbooks on genetics and molecular biology. Amino acid sequences which are produced from SEQ ID No. 2 in an appropriate manner are also a constituent of the invention.

In the same way, DNA sequences which hybridize with SEQ ID No. 1 or parts of SEQ ID No. 1 are a constituent of the invention. Finally, DNA sequences which are produced from SEQ ID No. 1 by the polymerase chain reaction (PCR) using primers are a constituent of the invention. These types of oligonucleotides typically have a length of at least 15 nucleotides.

Instructions for identifying DNA sequences by means of hybridization can be found by a person skilled in the art, inter alia, in the manual “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R. Hybridization takes place under stringent conditions, which means that the only hybrids formed are those in which the probe and target sequence, i.e. the polynucleotides treated with the probes, are at least 70% identical. It is known that the stringency of hybridization, including the washing step, is affected or determined by varying the buffer composition, the temperature and the salt concentration. The hybridization reaction is preferably performed at relatively low stringency as compared with the washing steps (Hybaid Hybridization Guide, Hybaid Limited, Teddington, UK, 1996 I.B.R.).

For the hybridization reaction, for example, a 5×SSC-buffer may be used at a temperature of about 50° C.-68° C. Probes may then also hybridize with polynucleotides which are less than 70% identical to the sequence in the probe. These hybrids are less stable and are removed by washing under stringent conditions. This may be achieved, for example, by lowering the salt concentration to 2×SSC and optionally then to 0.5×SSC (The DIG System User's Guide for Filter Hybridization, Boehringer Mannheim, Mannheim, Germany, 1995 I.B.R.), wherein a temperature of about 50° C.-68° C. is used. It is also optionally possible to lower the salt concentration to 0.1×SSC. By a stepwise increase in the hybridization temperature from 50° C. to 68° C., in steps of about 1-2° C., polynucleotide fragments can be isolated which are, for example, at least 70% or at least 80% or at least 90% to 95% identical to the sequence in the probe used. Further instructions for hybridization, in the form of so-called kits, are commercially available (e.g. DIG Easy Hyb from Roche Diagnostics GmbH, Mannheim, Germany, Catalogue No. 1603558).

A person skilled in the art may find instructions for the amplification of DNA sequences using the polymerase chain reaction (PCR), inter alia, in the manual by Gait: Oligonucleotide Synthesis: A Practical Approach (IRL Press, Oxford, UK, 1984) I.B.R. and in Newton and Graham: PCR (Spektrum Akademischer Verlag, Heidelberg, Germany, 1994) I.B.R.

It was found that coryneform bacteria produce amino acids in an improved manner following overexpression of the dctA gene.

To produce overexpression, the copy number of the corresponding gene may be increased, or the promoter and regulation region or the ribosome bonding site which is located upstream of the structure gene, is mutated. Expression cassettes which are incorporated upstream of the structure gene act in the same way. It is also possible to increase the expression during the course of fermentative amino acid production by inducible promoters. Expression is also improved by measures to extend the lifetime of mRNA. Furthermore, enzyme activity is also enhanced by preventing the degradation of the enzyme protein. The gene or gene constructs may either be present in plasmids with different copy numbers or integrated in the chromosome and amplified. Alternatively, furthermore, overexpression of the gene concerned may be achieved by changing the composition of the medium and culture management.

A person skilled in the art can find instructions for this, inter alia, in Martin et al. (Bio/Technology 5, 137-146 (1987)) I.B.R., in Guerrero et al. (Gene 138, 35-41 (1994)) I.B.R., in Tsuchiya and Morinaga (Bio/Technology 6, 428-430 (1988)) I.B.R., in Eikmanns et al. (Gene 102, 93-98 (1991)), in European patent 0 472 869 I.B.R., in U.S. Pat. No. 4,601,893 I.B.R., in Schwarzer and Pühler (Bio/Technology 9, 84-87 (1991) I.B.R., in Reinscheid et al. (Applied and Environmental Microbiology 60, 126-132 (1994)) I.B.R., in LaBarre et al. (Journal of Bacteriology 175, 1001-1007 (1993)) I.B.R., in patent application WO 96/15246 I.B.R., in Malumbres et al. (Gene 134, 15-24 (1993)) I.B.R., in Japanese patent document JP-A-10-229891 I.B.R., in Jensen and Hammer (Biotechnology and Bioengineering 58, 191-195 (1998)) I.B.R., in Makrides (Microbiological Reviews 60:512-538 (1996)) I.B.R. and in known textbooks on genetics and molecular biology.

By way of example, for enhancement purposes, the dctA gene according to the invention was overexpressed with the aid of episomal plasmids. Suitable plasmids are those which are replicated in coryneform bacteria. Many known plasmid vectors such as e.g. pZ1 (Menkel et al., Applied and Environmental Microbiology (1989) 64: 549-554 I.B.R.), pEKEx1 (Eikmanns et al., Gene 102:93-98 (1991) I.B.R.) or pHS2-1 (Sonnen et al., Gene 107:69-74 (1991) I.B.R.) are based on the cryptic plasmids pHM1519, pBL1 or pGA1. Other plasmid vectors such as e.g. those which are based on pCG4 (U.S. Pat. No. 4,489,160) I.B.R., or pNG2 (Serwold-Davis et al., FEMS Microbiology Letters 66, 119-124 (1990) I.B.R.), or pAG1 (U.S. Pat. No. 5,158,891 I.B.R.), may be used in the same way.

Furthermore, those plasmid vectors with the aid of which the process of gene amplification by integration in the chromosome can be applied are also suitable, as was described, for example, by Reinscheid et al. (Applied and Environmental Microbiology 60, 126-132 (1994)) I.B.R. for the duplication or amplification of the hom-thrB operon. In this method, the complete gene is cloned in a plasmid vector which can replicate in a host (typically E. coli), but not in C. glutamicum. Suitable vectors are, for example, pSUP301 (Simon et al., Bio/Technology 1, 784-791 (1983) I.B.R.), pK18mob or pK19mob (Schäfer et al., Gene 145, 69-73 (1994) I.B.R.), pGEM-T (Promega corporation, Madison, Wis., USA), pCR2.1-TOPO (Shuman (1994). Journal of Biological Chemistry 269:32678-84 I.B.R.; U.S. Pat. No. 5,487,993 I.B.R.), pCR®Blunt (Firma Invitrogen, Groningen, Netherlands; Bernard et al., Journal of Molecular Biology, 234: 534-541 (1993) I.B.R.), pEM1 (Schrumpf et al, 1991, Journal of Bacteriology 173:4510-4516 I.B.R.) or pBGS8 (Spratt et al., 1986, Gene 41: 337-342 I.B.R.). The plasmid vector which contains the gene to be amplified is then transferred to the desired strain of C. glutamicum by conjugation or transformation. The method of conjugation is described, for example, in Schafer et al. (Applied and Environmental Microbiology 60, 756-759 (1994)) I.B.R. Methods for transformation are described, for example, in Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. Following homologous recombination by means of a “cross-over” event, the resulting strain contains at least two copies of the gene concerned.

In addition, it may be advantageous for the production of L-amino acids, in addition to enhancing the dctA gene in one or more enzymes on the relevant biosynthetic pathway, to enhance, in particular overexpress, glycolysis, anaploretic processes, the citric acid cycle, the pentose-phosphate cycle, amino acid export and optionally regulatory proteins.

Thus for example to prepare L-amino acids, in addition to enhancing the dctA gene, one or more of the genes chosen from the group

the dapA gene coding for dihydrodipicolinate synthase (EP-B 0 197 335 I.B.R.),

the gap gene coding for glyceraldehyde-3-phosphate dehydrogenase (Eikmanns (1992), Journal of Bacteriology 174:6076-6086 I.B.R.),

the tpi gene coding for triosephosphate isomerase (Eikmanns (1992), Journal of Bacteriology 174:6076-6086 I.B.R.),

the pgk gene coding for 3-phosphoglycerate kinase (Eikmanns (1992), Journal of Bacteriology 174:6076-6086 I.B.R.),

the zwf gene coding for glucose-6-phosphate dehydrogenase (JP-A-09224661 I.B.R.),

the pyc gene coding for pyruvate carboxylase (DE-A-198 31 609 I.B.R.),

the mqo gene coding for malate quinone oxidoreductase (Molenaar et al., European Journal of Biochemistry 254, 395-403 (1998) I.B.R.),

the lysC gene coding for a feed-back resistant aspartate kinase (Accession no. P26512; EP-B-0387527 I.B.R.; EP-A-0699759 I.B.R.),

the lysE gene coding for lysine export (DE-A-195 48 222 I.B.R.),

the hom gene coding for homoserine dehydrogenase (EP-A 0131171 I.B.R.),

the ilvA gene coding for threonine dehydratase (Möckel et al., Journal of Bacteriology (1992) 8065-8072)) or the ilvA (Fbr) allele coding for a “feed back resistant” threonine dehydratase (Möckel et al., (1994) Molecular Microbiology 13: 833-842 I.B.R.),

the ilvBN gene coding for acetohydroxyacid synthase (EP-B 0356739 I.B.R.),

the ilvD gene coding for dihydroxyacid dehydratase (Sahm and Eggeling (1999) Applied and Environmental Microbiology 65: 1973-1979 I.B.R.),

the zwa1 gene coding for Zwa1 protein (DE: 19959328.0 I.B.R., DSM 13115)

may be simultaneously enhanced, in particular overexpressed.

Furthermore, it may also be advantageous for the production of L-amino acids, apart from enhancing the dctA gene, to simultaneously attenuate, in particular to reduce the expression of, one or more genes chosen from the group

the pck gene coding for phosphoenolpyruvate carboxykinase (DE 199 50 409.1 I.B.R.; DSM 13047),

the pgi gene coding for glucose-6-phosphate isomerase (U.S. Ser. No. 09/396,478 I.B.R.; DSM 12969),

the poxB gene coding for pyruvate oxidase (DE: 1995 1975.7 I.B.R.; DSM 13114)

the zwa2 gene coding for Zwa2 protein (DE: 199 59 327.2 I.B.R., DSM 13113).

The expression “attenuation” in this context describes the reduction in or switching off of the intracellular activity of one or more enzymes (proteins) in a microorganism, which are coded by the corresponding DNA, by for example using a weak promoter or using a gene or allele which codes for a corresponding enzyme with a lower activity or inactivates the corresponding gene or enzyme (protein) and optionally by combining these measures.

As a result of attenuation measures, the activity or concentration of the corresponding protein is generally lowered to 0 to 75%, 0 to 50%, 0 to 25%, 0 to 10% or 0 to 5% of the activity or concentration of the wild type protein or of the activity or concentration of the protein in the initially used microorganism.

Furthermore, it may be advantageous for the production of amino acids, apart from overexpressing the dctA gene, to switch off undesired side reactions (Nakayama: “Breeding of Amino Acid Producing Microorganisms”, in: Overproduction of Microbial Products, Krumphanzl, Sikyta, Vanek (eds.), Academic Press, London, UK, 1982 I.B.R.).

Microorganisms prepared according to the invention are also provided by the invention and may be cultivated continuously or batchwise in a batch process or in a fed batch process or repeated fed batch process for the purposes of producing amino acids. A review of known cultivation processes is given in the text book by Chmiel (Bioprozesstechnik 1. Einführung in die Bioverfahrenstechnik (Gustav Fischer Verlag, Stuttgart, 1991) I.B.R.) or in the textbook by Storhas (Bioreaktoren und periphere Einrichtungen (Vieweg Verlag, Braunschweig/Wiesbaden, 1994) I.B.R.).

The culture medium to be used has to comply in a suitable manner with the requirements of the particular strain. Descriptions of culture media for different microorganisms are given in the manual “Manual of Methods for General Bacteriology” by the American Society for Bacteriology (Washington D.C., USA, 1981) I.B.R.

Sources of carbon which may be used are sugars and carbohydrates such as e.g. glucose, saccharose, lactose, fructose, maltose, molasses, starch and cellulose, oils and fats such as, for example, soya oil, sunflower oil, peanut oil and coconut oil, fatty acids such as, for example, palmitic acid, stearic acid and linoleic acid, alcohols such as, for example, glycerin and ethanol and organic acids such as, for example, acetic acid. These substances may be used individually or as a mixture.

Sources of nitrogen which may be used are organic nitrogen-containing compounds such as peptones, yeast extract, meat extract, malt extract, corn steep liquor, soya bean meal and urea or inorganic compounds such as ammonium sulfate, ammonium chloride, ammonium phosphate, ammonium carbonate and ammonium nitrate. The sources of nitrogen may be used individually or as a mixture.

Sources of phosphorus which may be used are phosphoric acid, potassium dihydrogen phosphate or dipotassium hydrogen phosphate or the corresponding sodium-containing salts. The culture medium must also contain salts of metals such as, for example, magnesium sulfate or iron sulfate, which are required for growth. Finally, essential growth-promoting substances such as amino acids and vitamins may be used in addition to the substances mentioned above. Suitable precursors may be added to the culture medium in addition to these. The feedstuffs mentioned may be added to the culture in the form of a single batch or be fed in a suitable manner during cultivation.

To regulate the pH of the culture, basic compounds such as sodium hydroxide, potassium hydroxide, ammonia or ammoniacal liquor or acid compounds such as phosphoric acid or sulfuric acid are used in an appropriate manner. To control the production of foam, antifoaming agents such as, for example, polyglycol esters of fatty acids may be used. To maintain the stability of plasmids, suitable selectively acting substances such as, for example, antibiotics, may be added to the medium. In order to maintain aerobic conditions, oxygen or oxygen-containing gas mixtures such as, for example, air, are passed into the culture. The temperature of the culture is normally 20° C. to 45° C. and is preferably 25° C. to 40° C. The culture procedure is continued until a maximum has been produced in the desired product. This objective is normally achieved within 10 hours to 160 hours.

Methods for determining L-amino acids are known from the prior art. Analysis may be performed, for example, as described in Spackman et al. (Analytical Chemistry, 30, (1958), 1190) I.B.R. by ion exchange chromatography followed by ninhydrin derivation, or it may be performed by reversed phase HPLC as described in Lindroth et al. (Analytical Chemistry (1979) 51: 1167-1174) I.B.R.

The process according to the invention is used for the fermentative preparation of amino acids.

The present invention is explained in more detail in the following by using working examples.

The following microorganism was deposited on 18th May 2001 as a pure culture at the German Collection of Microorganisms and Cell Cultures (DSMZ, Braunschweig, Germany) in accordance with the Budapest Treaty:

Escherichia coli DH5αmcr/pEC-XK99EdctAb1ex as DSM 14314.

Isolation of plasmid DNA from Escherichia coli and all the techniques for restriction, Klenow treatment and alkaline phosphatase treatment were performed in the way described in Sambrook et al. (Molecular Cloning. A Laboratory Manual, 1989, Cold Spring Harbor Laboratory Press, Cold Spring Harbor, N.Y., USA) I.B.R. Methods for the transformation of Escherichia coli are also described in this manual.

The composition of commonly used culture media such as LB medium or TY medium may also be found in the manual by Sambrook et al.

### EXAMPLE 1

### Production of a Genomic Cosmid Gene Library from Corynebacterium glutamicum ATCC 13032

Chromosomal DNA from Corynebacterium glutamicum ATCC 13032 was isolated as described in Tauch et al., (1995, Plasmid 33:168-179) I.B.R., and partly cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Diagnostics GmbH, Mannheim, Germany, product description SAP, Code no. 1758250). The DNA in the cosmid vector SuperCos1 (Wahl et al. (1987), Proceedings of the National Academy of Sciences, USA 84:2160-2164 I.B.R.), purchased from Stratagene (La Jolla, USA, product description SuperCos1 Cosmid Vektor Kit, Code no. 251301) was cleaved with the restriction enzyme XbaI (Amersham Pharmacia, Freiburg, Germany, product description XbaI, Code no. 27-0948-02) and also dephosphorylated with shrimp alkaline phosphatase.

Then the cosmid DNA was cleaved with the restriction enzyme BamHI (Amersham Pharmacia, Freiburg, Germany, product description BamHI, Code no. 27-0868-04). The cosmid DNA treated in this way was mixed with the treated ATCC13032 DNA and the mixture was treated with T4 DNA ligase (Amersham Pharmacia, Freiburg, Germany, product description T4-DNA-Ligase, Code no.27-0870-04). The ligation mixture was then packed into phages with the aid of Gigapack II XL Packing Extracts (Stratagene, La Jolla, USA, product description Gigapack II XL Packing Extract, Code no. 200217).

To infect E. coli strain NM554 (Raleigh et al. 1988, Nucleic Acid Research 16:1563-1575 I.B.R.), the cells were taken up in 10 mM MgSO₄ and mixed with an aliquot of the phage suspension. Infection and titering of the cosmid library were performed as described in Sambrook et al. (1989, Molecular Cloning: A Laboratory Manual, Cold Spring Harbor), wherein the cells were plated out on LB agar (Lennox, 1955, Virology, 1:190 I.B.R.)+100 mg/l ampicillin. After incubation overnight at 37° C., recombinant individual clones were selected.

### EXAMPLE 2

### Isolating and Sequencing the dctA Gene

The cosmid DNA from an individual colony was isolated with the Qiaprep Spin Miniprep Kit (Product No. 27106, Qiagen, Hilden, Germany) in accordance with the manufacturer's information and partly cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Diagnostics GmbH, Mannheim, Germany, product description SAP, Product No. 1758250). After gel electrophoretic separation, isolation of the cosmid fragments in the size range 1500 to 2000 bp was performed with QiaExII Gel Extraction Kit (Product No. 20021, Qiagen, Hilden, Germany).

The DNA in sequencing vector pZero-1 purchased from Invitrogen (Groningen, Netherlands, product description Zero Background Cloning Kit, Product No. K2500-01) was cleaved with the restriction enzyme BamHI (Amersham Pharmacia, Freiburg, Germany, product description BamHI, Product No. 27-0868-04). Ligation of the cosmid fragments in sequencing vector pZero-1 was performed as described in Sambrook et al. (1989, Molecular Cloning: A Laboratory Manual, Cold Spring Harbor) I.B.R., wherein the DNA mixture was incubated overnight with T4 ligase (Pharmacia Biotech, Freiburg, Germany). This ligation mixture was then electropored in E. coli strain DH5αMCR (Grant, 1990, Proceedings of the National Academy of Sciences, U.S.A., 87:4645-4649 I.B.R.) (Tauch et al. 1994, FEMS Microbiol Letters, 123:343-7 I.B.R.) and plated out on LB agar (Lennox, 1955, Virology, 1:190 I.B.R.) with 50 mg/l zeocin.

The plasmid preparation of recombinant clones was performed with Biorobot 9600 (Product No. 900200, Qiagen, Hilden, Germany). Sequencing was performed using the dideoxy chain termination method according to Sanger et al. (1977, Proceedings of the National Academy of Sciences, U.S.A., 74:5463-5467) I.B.R. with modifications by Zimmermann et al. (1990, Nucleic Acids Research, 18:1067) I.B.R. The “RR dRhodamin Terminator Cycle Sequencing Kit” from PE Applied Biosystems (Product No. 403044, Weiterstadt, Germany) was used. Gel electrophoretic separation and analysis of the sequencing reaction was performed in a “Rotiphorese NF Acrylamid/Bisacrylamid” Gel (29:1) (Product No. A124.1, Roth, Karlsruhe, Germany) using the “ABI Prism 377” sequencing instrument from PE Applied Biosystems (Weiterstadt, Germany).

The crude sequencing data obtained were then processed using the Staden software package (1986, Nucleic Acids Research, 14:217-231 I.B.R.) Version 97-0. The individual sequences of the pZero1 derivatives were assembled to give a cohesive contig. Computer aided coding region analyses were drawn up with the program XNIP (Staden, 1986, Nucleic Acids Research, 14:217-231 I.B.R.). Further analyses can be carried out with the “BLAST search program” (Altschul et al., 1997, Nucleic Acids Research, 25:3389-3402 I.B.R.) against the non-redundant databank of the “National Center for Biotechnology Information” (NCBI, Bethesda, Md., USA) I.B.R.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The nucleotide sequence obtained is given in SEQ ID No. 1. Analysis of the nucleotide sequence produced an open reading frame of 1341 bp, which was called the dctA gene. The dctA gene codes for a protein of 446 amino acids.

### EXAMPLE 3

### Preparation of the Shuttle Expression Vector pEC-XK99EdctAb1ex for Enhancing the dctA Gene in C. glutamicum

Cloning the dctA Gene

Chromosomal DNA was isolated from the strain ATCC 13032 using the method described by Eikmanns et al. (Microbiology 140: 1817-1828 (1994)) I.B.R. On the basis of the sequence for the dctA gene, known for C. glutamicum from example 2, the following oligonucleotides were chosen for the polymerase chain reaction (see SEQ ID No. 3 and SEQ ID No. 4):

dctAex1:

5′ ca ggt acc-aag gtc gaa gag gaa gtt ca 3′ SEQ ID NO:3

dctAex2:

5′ ga tct aga-cgg ccg gac atg cat tgt at 3′ SEQ ID NO:4

The primers shown were synthesized by MWG-Biotech AG (Ebersberg, Germany) and the PCR reaction was performed using the standard PCR method described by Innis et al. (PCR protocols. A guide to methods and applications, 1990, Academic Press) I.B.R. using Pwo polymerase from Roche Diagnostics GmbH (Mannheim, Germany). With the aid of the polymerase chain reaction, the primers enabled amplification of an approximately 1.42 kb sized DNA fragment which contained the dctA gene. In addition, the primer dctAex1 contained the sequence for the restriction site of the restriction endonuclease KpnI, and the primer dctAex2 contained the restriction site for the restriction endonuclease XbaI, which are indicated by underlining on the nucleotide sequences shown above.

The approximately 1.42 kb sized dctA fragment was cleaved with the restriction endonucleases KpnI and XbaI and then isolated from the agarose gel with the QiaExII Gel Extraction Kit (Product No. 20021, Qiagen, Hilden, Germany).

Constructing the Shuttle Vector pEC-XK99E

The E. coli-C. glutamicum shuttle vector pEC-XK99E was constructed in accordance with the prior art. The vector contained the replication region rep from the plasmid pGA1 including the replication effector per (U.S. Pat. No. 5,175,108 I.B.R.; Nesvera et al., Journal of Bacteriology 179, 1525-1532 (1997)) I.B.R., the kanamycin resistance gene aph(3′)-IIa from Escherichia coli (Beck et al. (1982), Gene 19: 327-336 I.B.R.), the replication origin, the trc promoter, the termination regions T1 and T2, the lacI^{q} gene (repressor of the lac operon from E. coli) and a multiple cloning site (mcs) (Norrander, J. M. et al. Gene 26, 101-106 (1983) I.B.R.) from the plasmid pTRC99A (Amann et al. (1988), Gene 69: 301-315 I.B.R.).

The E. coli-C. glutamicum shuttle vector pEC-XK99E constructed was transferred into C. glutamicum DSM5715 by means of electroporation (Liebl et al., 1989, FEMS Microbiology Letters, 53:299-303 I.B.R.). The transformants were selected on LBHIS agar consisting of 18.5 g/l brain-heart infusion bouillon, 0.5 M sorbitol, 5 g/l bacto trypton, 2.5 g/l bacto yeast extract, 5 g/l NaCl and 18 g/l bacto agar, which had been supplemented with 25 mg/l kanamycin. Incubation was performed for 2 days at 33° C.

Plasmid DNA was isolated from a transformant by the conventional method (Peters-Wendisch et al., 1998, Microbiology, 144, 915-927) I.B.R., restricted with the restriction endonuclease HindIII and the plasmid was examined by subsequent agarose gel electrophoresis.

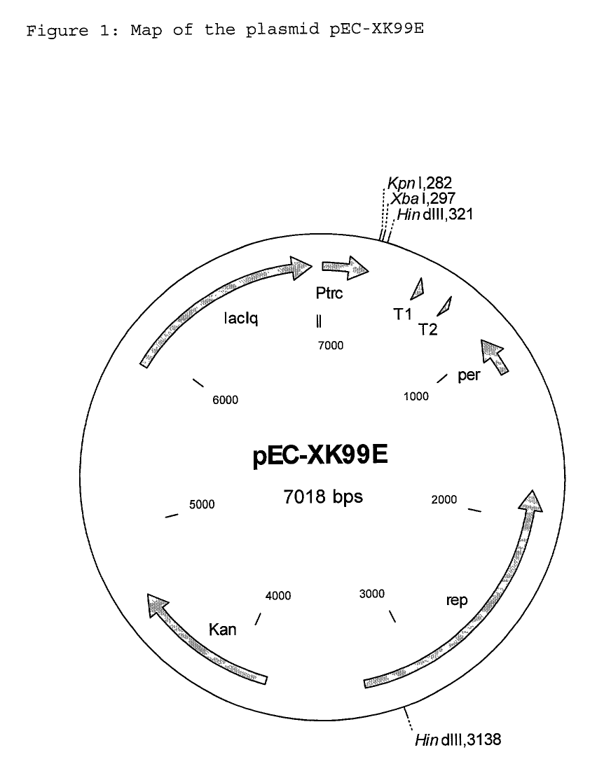

The plasmid construct obtained in this way was called pEC-XK99E (FIG. 1). The strain obtained by electroporation of the plasmid pEC-XK99E in C. glutamicum strain DSM5715 was called DSM5715/pEC-XK99E and, as DSM13455, was deposited at the German Collection of Microorganisms and Cell Cultures (DSMZ, Braunschweig, Germany) in accordance with the Budapest Treaty.

Cloning of dctA in the E. coli-C. glutamicum Shuttle Vector pEC-XK99E

The E. coli-C. glutamicum shuttle vector pEC-XK99E described in example 3.2 was used as the vector. DNA from this plasmid was completely cleaved with the restriction enzymes KpnI and XbaI and then dephosphorylated with shrimp alkaline phosphatase (Roche Diagnostics GmbH, Mannheim, Germany, product description SAP, Product No. 1758250).

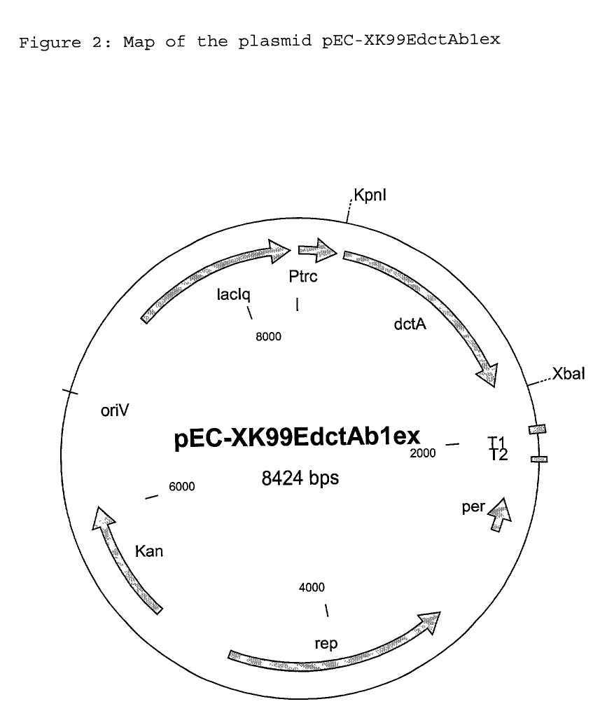

The 1.4 kb sized dctA fragment described in example 3.1, obtained by means of PCR and cleaved with the restriction endonucleases KpnI and XbaI was mixed with the previously prepared vector pEC-XK99E and the mixture was treated with T4 DNA ligase (Amersham Pharmacia, Freiburg, Germany, product description T4-DNA-Ligase, Code no.27-0870-04). The ligation mixture was transformed in E. coli strain DH5αmcr (Hanahan, In: DNA cloning. A practical approach. Vol. I. IRL-Press, Oxford, Washington D.C., USA I.B.R.). The plasmid-containing cells were selected by plating out the transformation mixture on LB agar (Lennox, 1955, Virology, 1:190) I.B.R. with 50 mg/l kanamycin. Following incubation overnight at 37° C., recombinant individual clones were selected. Plasmid DNA was isolated from a transformant using the Qiaprep Spin Miniprep Kit (Product No. 27106, Qiagen, Hilden, Germany) in accordance with the manufacturer's information and cleaved with the restriction enzymes KpnI and XbaI, in order to examine the plasmid by subsequent agarose gel electrophoresis. The plasmid obtained was called pEC-XK99EdctAb1ex. It is shown in FIG. 2.

### EXAMPLE 4

### Transformation of Strain DSM5715 Using the Plasmid pEC-XK99EdctAb1ex

Strain DSM5715 was transformed with plasmid pEC-XK99EdctAb1ex by applying the electroporation method described by Liebl et al., (FEMS Microbiology Letters, 53:299-303 (1989)) I.B.R. The transformants were selected on LBHIS agar consisting of 18.5 g/l brain-heart infusion bouillon, 0.5 M sorbitol, 5 g/l bacto trypton, 2.5 g/l bacto yeast extract, 5 g/l NaCl and 18 g/l bacto agar, which had been supplemented with 25 mg/l kanamycin. Incubation was performed for 2 days at 33° C.

Plasmid DNA was isolated from a transformant by the conventional methods (Peters-Wendisch et al., 1998, Microbiology, 144, 915-927 I.B.R.), cleaved with the restriction endonucleases KpnI and XbaI the plasmid was examined later by agarose gel electrophoresis. The strain obtained was called DSM5715/pEC-XK99EdctAb1ex1.

### EXAMPLE 5

### Preparing Lysine

The C. glutamicum strain DSM5715/pEC-XK99EdctAb1ex obtained in example 4 was cultivated in a nutrient medium suitable for the production of lysine and the lysine concentration in the culture supernatant liquid was determined.

For this purpose, the strain was first incubated for 24 hours at 33° C. on agar plates with the corresponding antibiotic (brain-heart agar with kanamycin (25 mg/l)). Starting with these agar plate cultures, a preculture was inoculated (10 ml of medium in 100 ml conical flasks). The complete medium CgIII was used as the medium for the preculture.

Medium Cg III

| | | |
|---|---|---|
| NaCl | 2.5 | g/l |
| Bacto peptone | 10 | g/l |
| Bacto yeast extract | 10 | g/l |
| Glucose (autoclaved separately) | 2% | (w/v) |
| The pH was adjusted to pH 7.4 | | |

Kanamycin (25 mg/l) was added to this. The preculture was incubated on the shaker at 33° C. for 16 hours at 240 rpm. A main culture was inoculated with this preculture so that the initial OD (660 nm) of the main culture was 0.1. The medium MM was used for the main culture.

Medium MM

| | | |
|---|---|---|
| CSL (Corn Steep Liquor) | 5 | g/l |
| MOPS (morpholinopropanesulfonic | 20 | g/l |
| acid) | | |
| Glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (filtered sterile) | 0.3 | mg/l |
| Thiamine * HCl (filtered sterile) | 0.2 | mg/l |
| Leucine (filtered sterile) | 0.1 | g/l |
| CaCO₃ | 25 | g/l |

CSL, MOPS and the salt solution are adjusted to pH 7 with ammoniacal liquor and autoclaved. Then the sterile substrate solution and vitamin solution, and also the dry-autoclaved CaCO₃ are added.

Cultivation takes place in 10 ml volumes in a 100 ml conical flask with baffles. Kanamycin (25 mg/l) was added. Cultivation takes place at 33° C. and 80% atmospheric humidity.

After 48 hours, the OD was determined at a test wavelength of 660 nm using the Biomek 1000 (Beckmann Instruments GmbH, Munich). The amount of lysine produced was determined with an amino acid analyzer from Eppendorf-BioTronik (Hamburg, Germany) by ion exchange chromatography and post-column derivation with ninhydrin detection.

Table 1 gives the results of the trial.

TABLE 1

| | OD | Lysine-HCl |
|---|---|---|
| Strain | (660 nm) | g/l |
| DSM5715 | 11.3 | 13.02 |
| DSM14314 | 12 | 14.02 |

This application claims priority to German Priority Document Application No. 100 46 230.8, filed on Sep. 19, 2000 and to German Priority Document Application No. 101 32 724.2, filed on Jul. 5, 2001. Both German Priority Documents are hereby incorporated by reference in their entirety.

We claim:

1. An isolated polynucleotide from coryneform bacteria, comprising a polynucleotide sequence coding for the dctA gene, selected from the group consisting of:
a) a polynucleotide which is at least 70% identical to a polynucleotide which codes for a polypeptide which contains the amino acid sequence in SEQ ID No. 2,
b) a polynucleotide which codes for a polypeptide which contains an amino acid sequence which is at least 70% identical to the amino acid sequence in SEQ ID No. 2,
c) a polynucleotide which is complementary to the polynucleotides in a) or b), and
d) a polynucleotide containing of at least 15 consecutive nucleotides from the polynucleotide sequence in a), b) or c).

2. The polynucleotide according to claim 1, wherein the polypeptide has C4 dicarboxylate transport protein activity.

3. The polynucleotide according to claim 1, wherein the polynucleotide is a recombinant DNA replicable in coryneform bacteria.

4. The polynucleotide according to claim 1, wherein the polynucleotide is an RNA.

5. The polynucleotide according to claim 3, comprising the nucleic acid sequence shown in SEQ ID No. 1.

6. The polynucleotide according to claim 3, wherein the DNA, comprises
(i) the nucleotide sequence shown in SEQ ID No. 1, or
(ii) at least one sequence which corresponds to sequence (i) within the range of the degeneracy of the genetic code, or
(iii) at least one sequence which hybridizes with the sequence which is complementary to sequence (i) or (ii).

7. The polynucleotide according to claim 6, further comprising
(iv) functionally neutral sense mutations in (i).

8. The polynucleotide according to claim 6, wherein the hybridization of sequence (iii) is carried out under conditions of stringency corresponding at most to 2×SSC.

9. The polynucleotide according to claim 1, wherein the polynucleotide codes for a polypeptide that comprises the amino acid sequence shown in SEQ ID NO: 2.

10. A coryneform bacteria, in which the dctA gene is enhanced.

11. The coryneform bacteria according to claim 8, wherein dctA gene is overexpressed.

12. An Escherichia coli DH5αmcr/pEC-XK99EdctAb1ex, as DSM 14314.

13. A method for the fermentative preparation of L-amino acids in coryneform bacteria comprising:
a) fermenting, in a medium. the desired L-amino acid-producing coryneform bacteria, in which at least
b) the dctA gene or nucleotide sequences coding for this gene are enhanced.

14. The method according to claim 13, further comprising:
b) concentrating the L-amino acid in the medium or in the cells of the bacteria.

15. The method according to claim 14, further comprising:
c) isolating the L-amino acid.

16. The method according to claim 13, wherein the L amino acids are lysine.

17. The method according to claim 13, wherein dctA gene or nucleotide sequences coding for this gene are overexpressed

18. The method according to claim 13, wherein additional genes of the biosynthesis pathway of the desired L-amino acid are enhanced in the bacteria.

19. The method according to claim 13, wherein bacteria in which metabolic pathways which reduce the formation of the desired L-amino acid are at least partly switched off.

20. The method according to claim 13, wherein a strain which is transformed with a plasmid vector is used and the plasmid vector contains a nucleotide sequence coding for the dctA gene.

21. The method according to claim 13, wherein expression of the polynucleotide(s) which code(s) for the dctA gene are enhanced.

22. The method according to claim 21, wherein expression of the polynucleotide(s) which code(s) for the dctA gene are overexpressed.

23. The method according to claim 13, wherein the catalytic properties of the polypeptide for which polynucleotide dctA codes are increased.

24. The method according to claim 13, wherein the bacteria fermented comprise, at the same time, one or more genes which are enhanced or overexpressed; wherein the one or more genes is/are selected from the group consisting of:
the dapA gene coding for dihydrodipicolinate synthase,
the gap gene coding for glyceraldehyde-3-phosphate dehydrogenase,
the tpi gene coding for triosephosphate isomerase,
the pgk gene coding for 3-phosphoglycerate kinase,
the zwf gene coding for glucose-6-phosphate dehydrogenase,
the pyc gene coding for pyruvate carboxylase,
the mqo gene coding for malate quinone oxidoreductase,
the lysC gene coding for feed-back resistant aspartate kinase,
the lysE gene coding for lysine export,
the hom gene coding for homoserine dehydrogenase,
the ilvA gene coding for threonine dehydratase or the ilVa(Fbr) allele coding for feed back resistant threonine dehydratase,
the ilvBN gene coding for acetohydroxyacid synthase,
the ilvD gene coding for dihydroxyacid dehydratase, and
the zwa1 gene coding for Zwa1 protein.

25. The method according to claim 13, wherein the bacteria fermented comprise, at the same time, one or more genes which are attenuated; wherein the genes are selected from the group consisting of:
the pck gene coding for phosphoenolpyruvate carboxykinase,
the pgi gene coding for glucose-6-phosphate isomerase,
the poxB gene coding for pyruvate oxidase, and
the zwa2 gene coding for Zwa2 protein.

26. The method according to claim 13, wherein microorganisms from the species Corynebacterium glutamicum are used.

27. The method according to claim 26, wherein the Corynebacterium glutamicum strain DH5αmcr/pEC-XK99EdctAb1ex is used.

28. A coryneform bacteria comprising a vector which contains a polynucleotide in accordance with claim 1.

29. A method for finding RNA, cDNA and DNA in order to isolate nucleic acids, polynucleotides or genes, which code for the C4 dicarboxylate transport protein or have a high similarity to the sequence in the dctA gene, comprising contacting the RNA, cDNA, or DNA with hybridization probes comprising polynucleotide sequences according to claim 1.

30. The method according to claim 29, wherein arrays, micro-arrays or DNA chips are used.
